(19) United States
(12) Patent Application Publication
(10) Pub. No.: US 2017/0298178 A1
(43) Pub. Date: Oct. 19, 2017

(54) DERIVATIVES OF 1,3-PROPANEDIOL

(71) Applicant: VANTAGE SPECIALTY CHEMICALS, INC., GURNEE, IL (US)

(72) Inventors: Eric J. Lind, Naperville, IL (US); Patricia A. Mayer, Bordentown, NJ (US); John A. Chase, Lebanon, NJ (US); Christine S. Fouts, East Palatka, FL (US)

(73) Assignee: VANTAGE SPECIALTY CHEMICALS, INC., GURNEE, IL (US)

(21) Appl. No.: 15/510,531

(22) PCT Filed: Sep. 14, 2015
(86) PCT No.: PCT/US15/50050

§ 371 Date: Mar. 10, 2017

Related U.S. Application Data
(60) Provisional application No. 62/050,009, filed on Sep. 12, 2014.

(51) Int. Cl. C08G 65/34; A61K 8/02; A61K 8/04; C07C 41/09

(52) U.S. Cl. CPC C08G 65/34; C07C 41/09; A61K 8/02; A61K 8/04

(57) ABSTRACT

The present invention relates to products that are derived from renewable biobased 1,3-propanediol (“1,3-PDO”). In this regard, aspects of the present invention involve the use of “green chemistry” in that products of the present invention are capable of being made from a renewable source of raw material.

This application is a U.S. National Stage application of PCT Application No. PCT/US2015/050050, filed Sep. 14, 2015, which application claims the benefit of priority to U.S. Provisional Application No. 62/050,009, filed Sep. 12, 2014, the disclosures of which are hereby incorporated herein by reference in their entirety.

The present invention relates to products that are derived from renewable biobased 1,3-propanediol (“1,3-PDO”). In this regard, aspects of the present invention involve the use of “green chemistry” in that products of the present invention are capable of being made from a renewable source of raw material.

There are numerous advantages that flow from the use of renewable 1,3-propanediol and derivatives thereof as a starting material for the preparation of emulsifying agents which have a combination of highly desirable properties. The 1,3-propanediol monomer may be derived from renewable resources such as corn by the fermentation of corn syrup. In aspects of the invention, polymers which are derived from 1,3-PDO offer advantages over alkoxylated agents in the preparation of emulsifiers in that the presence of 1,4-dioxane or residual ethylene oxide which is associated with their synthesis can, if desired, be avoided. Other advantages associated with the present invention are described below.

### SUMMARY OF THE INVENTION

In accordance with the present invention, there is provided a product prepared by reacting: (A) 1,3-propanediol or poly(1,3 propanediol) with (B) a fatty acid having a carbon chain length of about 2 to about 40 carbon atoms (hereafter “fatty acid reactant” or simply as “fatty acid”) to provide a product that is an ester of the fatty acid.

The invention also provides a product prepared by reacting 1,3-propanediol with glycerol and the use thereof to prepare a fatty acid ester by reacting the product with the fatty acid.

In addition, the invention includes within its scope a product prepared by reacting (A) poly(1,3-propanediol) with (B) polyglycerol to provide a poly(1,3-propanediolglycerol) copolymer that in turn can be reacted with the fatty acid reactant of above to provide a product that is an ester of the fatty acid.

Still another aspect of the present invention is the provision of a product prepared by reacting (A) 1,3 propanediol with (B) polyglycerol to provide a 1,3-propanediol capped polyglycerol that in turn can be reacted with the fatty acid reactant to produce an ester of the fatty acid.

Fatty acid esters identified above can be used as emulsifying agents in emulsions, that is, a composition comprising liquid droplets dispersed within a continuous phase of another liquid. As described below, various emulsions are improved by virtue of adding thereto fatty acid esters hereof, which for convenience, are also referred to herein as “E-additives.” Emulsions which are known in the art as “personal care formulations” are examples of emulsions which are benefitted by the presence of the E-additive.

### DETAILED DESCRIPTION OF THE INVENTION

As summarized above, the present invention includes the production of five classes of esters, each class including an ester prepared from the fatty acid reactant. The source of the “alcohol” reactant which forms the ester comprises 1,3-propanediol, poly(1,3-propanediol), 1,3 propanediol glycerol, poly(1,3-propanediol glycerol) copolymer or 1,3-propanediol capped polyglycerol.

The 1,3-“propanediol” reactants can be biobased and made from renewable resources, for example, corn. The fatty acid esters may be monoesters, diesters, triesters, tetraesters or combinations thereof.

Exemplary esters that can be prepared in accordance with the present invention can be represented by the structural diagram shown below.

R₁—C—O-Q-R₂ Structure 1

In Structure 1 above, R₁ or R₂ may be a substituted or unsubstituted aromatic, a saturated aliphatic, an unsaturated aliphatic or cycloaliphatic organic group containing from about 1 to about 40 carbons. For example, R₁ can be an aliphatic chain containing from about 6 to about 22 carbons and R₂ can be H or R₃CO, with R₃ being an aliphatic chain containing from about 6 to about 22 carbons. An example of Q is trimethylene or a residue of a polytrimethylene ether glycol after abstraction of the hydroxyl groups

Examples of fatty acids that can be used in making the esters of Structure 1 are stearic acid, coco fatty acid, oleic acid, behenic acid, and combinations thereof.

There follows an exemplary description of a fatty acid ester that is prepared by reacting 1,3-propanediol and a 2/40 fatty acid reactant at a temperature of between about 130° F. and about 200° F. at atmospheric pressure until the fatty acid reactant is in solution. (At room temperature the fatty acid reactant can range in form from a liquid to a solid.) 1,3-PDO is a colorless viscous liquid that is miscible with water at room temperature. The mole ratio of the fatty acid reactant to the 1,3-propanediol can be about 1:1 to about 2:1. The invention includes also within its scope diesters, triesters, and tetraesters of such esters. The mixture of reactants may be charged with at least one catalyst, preferably sodium hydroxide, phosphorous acid, and combinations thereof.

The mixture may be stirred with nitrogen purge at a temperature of about 180° F. for a time of about 20 minutes to about 30 minutes. The mixture can then be heated at a temperature of about 300° F. to about 450° F. for a time of about 5 hr to about 15 hr before being cooled to a temperature of about 150° F. to about 200° F. The mixture may then be neutralized to a pH of about 6.5 to about 7.5 with an acid, preferably phosphoric acid, methane sulfonic acid, and combinations thereof. The solid product produced by the reaction may be separated from the mixture by filtration.

Set forth below is a structural diagram of 1,3-propanediol stearate, an exemplary 1,3-propanediol fatty acid monoester of the type that can be prepared by the above reaction.

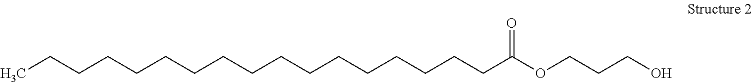 [SMILES: CCCCCCCCCCCCCCCCCC(=O)OCCCO]

In another embodiment of the present invention, the alcohol reactant is a poly(1,3-propanediol) that is reacted with the fatty acid reactant to provide a fatty acid ester of the present invention. Examples of poly(1,3-propanediols) are disclosed in U.S. Pat. No. 6,977,291 as are methods for their preparation.

Polymerized 1,3-propanediol can comprise about 1 to about 50 monomers, preferably about 1 to about 15 monomers. Preferably, the polymers have a molecular weight of less than about 2,500 Daltons.

The polymers can be used as intermediates in making fatty acid esters according to the present invention. Accordingly a poly(1,3-propanediol) can be reacted with a fatty acid reactant to produce a fatty acid ester that has surfactant properties, for example, a hydrophilic-lipophilic balance (HLB) from about 1 to about 8. In such a reaction, the mole ratio of the acid reactant to the alcohol reactant can be about 1 to 1 to about 8 to

Such fatty acid esters of the present invention can be prepared, for example, by mixing poly(1,3-propanediol) and a fatty acid reactant under conditions which are the same as described above in connection with the fatty acid ester made by reacting 1,3-propanediol with the fatty acid reactant.

Set forth below is a structural diagram of poly(1,3-propanediol) stearate which is an exemplary poly(1,3-propanediol) fatty acid ester.

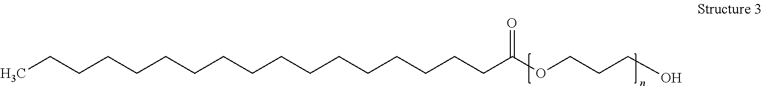

In the above structure, n can be, for example, 1 to about 50, preferably from 1 to about 15. The invention includes within its scope, for example, monoesters and diesters and mixture of such esters.

In still another embodiment of the invention, the alcohol for preparing the fatty acid ester can be prepared by reacting 1,3-propane glycol with triglycerol. This can be accomplished by heating the reactants in the presence of a catalyst, such as, for example, sodium hydroxide to 300-450° F. (as in preparation of poly(1,3 propanediol glycerol) copolymer as described below.

The aforementioned “1-3 propanediol/glycerol” can be reacted with a fatty acid reactant to form a fatty acid ester by, for example, heating the reactants to 300-450° F. in the presence of a catalyst such as sodium hydroxide.

In another embodiment of the present invention, the alcohol reactant for preparing the fatty acid ester is prepared by reacting poly(1,3-propanediol) with polyglycerol, preferably triglycerol, to provide a poly(1,3-propanediolglycerol) copolymer, which, for example, can be a solid product which is water soluble. Preferably, the copolymer has a molecular weight of less than about 2,500 Daltons.

The copolymer can be prepared, for example, by reacting poly(1,3-propanediol) with polyglycerol in the presence of a catalyst, preferably comprising sodium hydroxide. The reaction mixture is stirred with nitrogen purge at about room temperature for a time of about 20 minutes to about 30 minutes. The reaction mixture is then heated at a temperature of about 300° F. to about 400° F. for a time of about 5 hours to about 20 hours, before being cooled to about room temperature. The liquid poly(1,3-propanediolglycerol) copolymer may be subjected to further chemical reactions.

A fatty acid ester of the present invention can be prepared by reacting: a poly(1,3-propanediolglycerol) copolymer, as described above, with a fatty acid reactant to produce an ester of the fatty acid. The (poly(1,3-propanediolglycerol) copolymer and the fatty acid reactant are mixed at a temperature between about 130° F. and about 200° F. until the fatty acid is in solution. The reaction mixture may be charged with at least one catalyst selected from sodium hydroxide, phosphoric acid, phosphorous acid, and combinations thereof. The reaction mixture may be stirred with nitrogen purge at a temperature of about 180° F. for a time of from about 20 minutes to about 30 minutes. The temperature of the reaction mixture is then raised to a temperature of from about 300° F. to about 450° F. , and the reaction mixture is heated for a time of about 1 hour to about 10 hours, then cooled to a temperature of about 130° F. to about 200° F. The reaction mixture may then be neutralized to a pH of about 6.5 to about 7.5, preferably with an acid selected from the group consisting of methane sulfonic acid, phosphoric acid, and combinations thereof. The solid reaction product can be separated from the reaction mixture by filtration. Surfactant properties of the fatty acid ester can fall, for example, within the range of about 10 to about 20 HLB.

In another embodiment of the invention, a 1,3-propanediol capped polyglycerol is prepared by reacting (A) 1,3-propanediol with (B) polyglycerol. The 1,3-propanediol capped polyglycerol product may be used to prepare a fatty acid ester.

The 1,3-propanediol is reacted with polyglycerol (preferably, in a 1:1 mole ratio) in the presence of at least one catalyst, preferably sodium hydroxide. The reaction may be stirred with nitrogen purge at about room temperature for a time of about 20 minutes to about 30 minutes. The reaction is heated at a temperature of about 300° F. to about 400° F. for a length of time of about 1 to about 10 hours, then is cooled to about room temperature.

The 1,3-propanediol capped polyglycerol can be used to make a fatty acid ester by reacting it with a fatty acid reactant. Such fatty acid esters have surfactant properties, for example, an HLB of from about 8 to about 20, or from about 8 to about 13, a “High-HLB E-additive”.

By way of example, a 1,3-propanediol capped triglycerol can be reacted with a fatty acid reactant, preferably in about a 1:1 to about 1:2 mole ratio, at a temperature of about 130° F. to about 200° F., and mixed until all of the fatty acid is in solution, then purged with nitrogen for a time of about 20 minutes to about 30 minutes. The reaction mixture is then charged with at least one catalyst, preferably selected from sodium hydroxide, phosphoric acid, phosphorous acid, and combinations thereof. The reaction mixture heated for about 1 to about 20 hours at a temperature of about 300° F. to about 400° F., then cooled to a temperature of about 130° F. to about 200° F.

Set forth below is a structural diagram that represents exemplary fatty acid esters that fall within the scope of the present invention.

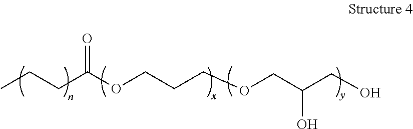

In preferred form, n may be from 0 to 38, x may be from 1 to 15, and y may be from 1 to about 30. The esters can include, for example, mono-, di-, tri-, tetra-esters, or a mixture of two or more thereof.

In accordance with the present invention, there can be prepared fatty acid esters which have surfactant properties that vary over a wide range, for example, with an HLB of about 1 to about 20. It is believed that those which have an HLB of about 1 to about 16 will be used most widely. For convenience, an E-additive which has an HLB of about to about 8 is referred to herein as a “Low-HLB E-additive” and one which has an HLB of about 8 to about 13 is referred to herein as a “High-HLB E-additive.”

As referred to above, the present invention includes within its scope the incorporation of a fatty acid ester hereof into an emulsion which typically comprises two or more liquids that are normally immiscible. An example of an emulsion is one in which droplets of water are dispersed in a continuous liquid phase of oil (water-in-oil emulsion). Another example of an emulsion is one in which droplets of an organic liquid are dispersed in a continuous water phase (oil-in-water emulsion). Ideally, the dispersed liquid droplets of an emulsion should be uniformly dispersed in the continuous liquid phase and they should remain uniformly dispersed if the emulsion is to remain stable. By way of background, it should be understood that immiscible liquids typically tend to separate into layers and an emulsion thereof does not form spontaneously; for example, energy input through vigorous mixing is required. Once formed, many emulsions tend to destabilize.

Emulsifying agents (also referred to commonly as an “emulsifier”) are critical to imparting stability to an emulsion which otherwise tends to destabilize. Various theories have been proposed as to how emulsifying agents function, for example: (A) by reducing the interfacial tension between the dispersed liquid and continuous liquid phase; and (B) by forming a film over the dispersed phase which causes the dispersed droplets to repel each other—this aids in maintaining them in suspension and improves the stability of the emulsion; and by (C) increasing the viscosity of the continuous phase; this aids in maintaining the dispersed phase in suspension. It is believed (but not confirmed) that the emulsifying agent of the present invention helps to maintain the stability of emulsions which contain them as additives by functioning in the various ways referred to in aforementioned (A), (B) and (C). It is noted that E-additives of the present invention can function as thickeners.

The art recognizes that emulsions can exist in various forms, for example, solid, semi-solid, and liquid dispersions or suspensions. Emulsifying agents of the present invention are capable of not only forming immiscible liquids into liquid compositions, but also into other forms of compositions, as exemplified above, and rendering them stable. Observations have shown that the fatty acid esters and personal care formulations hereof have excellent stability, for example, more than one year.

Although the emulsifying agents of the present invention can be used in a variety of different types of emulsions, their use in recommended particularly in emulsions comprising personal care formulations, many of which are typically oil-in-water emulsions. Speaking generally, such emulsions comprise products that are used in personal hygiene and for beautification, for example, cosmetics. Such products include, for example, creams, moisturizers, shampoos, hair conditioners; various skin care products such as body care lotions, facial and hand-care compositions; sunscreen, make-up remover, and liquid soaps.

Such emulsions comprise typically a major amount of water (for example, at least about 50 wt. %, typically about 65 to about 85 wt. %) as the continuous phase and other liquid ingredients dispersed therein. The use of such ingredients can vary depending on the particular personal care formulation involved. For example, an emollient is a widely used ingredient in such formulations where it functions, for example, to impart to the external layers of skin, or mucous membrane a soothing and softening effect, including soothing irritations in the skin. Emollients include hydrating agents comprising fat or oil that can be applied topically to soften the skin. Emollients are effective also in preventing and treating dry skin by functioning as a moisturizer; they keep aging skin soft and well hydrated.

Examples of emollients are tridecyl stearate, neopentyl glycol dicaprylate/dicaprate, tridecyl trimellitate, and mixtures thereof. There can be used also natural emollients, for example, castor oil, grape seed oil, and oils from sources such as avocados, apricots, coconuts, jojoba seeds, and crambe plants. Other examples of emollients, including synthetic compounds, are disclosed in the Example section hereof. The amount of emollient should comprise an amount which is effective to accomplish its function, for example, an amount of about 1 to about 20 wt. % of the emulsion.

The amount of the E-additive for use in the present invention will depend on various factors, for example, the particular personal care formulation and particular fatty acid ester being used; it should be used in an amount effective to accomplish its function. A preferred amount falls within the range of about 0.1 to about 10 wt. % of the emulsion; there may be applications where as much as 25 wt. % of the E-additive may be used.

The emulsions can contain other ingredients also, for example, preservatives, humectants, chelants, and thickeners. If present, they can comprise up to about 10 wt. % of the emulsion.

There follow general guidelines in the use of E-additives in the emulsions hereof. For example, in one embodiment of the invention, can be a mixture of E-additives, for example, at least one having a low-HLB and at least one having a high-HLB. The use of this technique can be used to adjust the viscosity of the emulsion, for example, to provide a lotion having a viscosity of less than 85,000 cp or to provide a cream that has a viscosity greater than about 85,000 cp.

The viscosity of the emulsions can vary over a wide range, for example, from about 2,000 cp to about 300,000 cp. It is believed that a good many emulsions will have a viscosity within the range of about 10,000 to about 200,000 cp.

In preparing emulsions of the present invention, homogenizing techniques or standard stirring and mixing techniques can be used.

### Standard Stirring Procedure

1) In a main vessel, distilled water and chelating agent are mixed with medium propeller mixing (approximately 350 rpm).
2) In a side vessel, glycerol is combined with a thickener, which preferably may be selected from xanthan gum, agar, carrageenan, fatty alcohols, and combinations thereof to make a slurry.
3) The slurry is added to the main vessel as mixing is continued.
4) The contents of the main vessel are heated to between about 70° F. and about 90° F. and are held at a temperature of between about 70° F. and about 90° F. for about 15-35 minutes while mixing.
5) In a secondary vessel, at least one E-additive surfactant is combined with a fatty alcohol thickener, emollient, and preservative.
6) The contents of the secondary vessel are heated to between about 70° F. and about 90° F., preferably about 80° F.
7) Thereafter the heated contents of the secondary vessel are slowly added to the main vessel with continued mixing.
8) After about 5 minutes, the mixture of the main vessel is removed from its heat source and slowly cooled while propeller mixing is continued.
9) When the temperature of the mixture reaches about 25° to about 35° F., mixing is stopped and the preparation of the emulsion is considered complete.

### Homogenizing Procedure

1) In a main vessel, distilled water and chelating agent are mixed with medium propeller mixing (350 rpm).
2) In a side vessel, glycerol is combined with a thickener which preferably may be selected from xanthan gum, agar, carrageenan, fatty alcohols, and combinations thereof to make a slurry.
3) The slurry is added to the main vessel with continued mixing.
4) The contents of the main vessel are heated to between about 70° F. and about 90° F. and are held at between about 70° F. and 90° F. for about 15-35 minutes while mixing.
5) In a secondary vessel, at least one E-additive surfactant is combined with a fatty alcohol thickener, emollient, and preservative.
6) The contents of the secondary vessel are heated to between about 70° F. and 90°

F., preferably about 80° F.
7) Thereafter, the heated contents of the secondary vessel are slowly added to the contents of the main vessel with continued mixing.
8) After about 5 minutes, the mixture of the secondary vessel is removed from its heat source and switched to a homogenizer and mixed at approximately 4000 rpm for about 3 minutes.
9) The mixture is then switched back to propeller mixing at about 350 rpm and then slowly cooled with continued mixing.
10) When temperature of the mixture reaches about 25° to about 35° F., mixing is stopped and the preparation of the emulsion is considered complete.

It should be understood that the above procedures are exemplary and that other methods can be used to make the emulsions.

The following examples serve to illustrate further the present invention.

### EXAMPLES

The Examples below describe the preparation of products according to the present invention and the preparation of compositions in which the products may be used.

Unless stated otherwise, “%” means—wt. %—and the conditions of reactions described in the examples were carried out at atmospheric pressure.

### Example 1

This example describes the synthesis of a polymer that may be used as an intermediate in the synthesis of a product which can be used as an emulsifying agent (E-additive) according to an embodiment of the present invention.

### Synthesis of Poly(1,3-propanediol)

A clean, dry 4-necked round bottom flask equipped with a condenser and overhead stirrer was charged with 1500 g of 1,3-propanediol (ZEMEA, available from DuPont Tate & Lyle Bio Products) and 1.92 g of 70% methane sulfonic acid (MSA). The condenser was cooled with tap water at 55-85° F. The resulting reaction mixture was stirred with nitrogen purge for 30 minutes at room temperature (about 73° F.). The reaction mixture was then heated to 360° F. and maintained at that temperature for 11 hours. The temperature of the reaction mixture was increased to 425° F. and the mixture was heated for an additional 5.5 hours at this temperature before being cooled to room temperature.

1192 grams of the resulting liquid poly(1,3-propanediol) product was collected from the reaction. The product had the following properties: gel permeation chromatography (GPC) Mp (peak molecular weight)=405, viscosity @25° C.=125 cp, refractive index=1.45477, and hydroxyl value=539 mg KOH/g.

### Example 2

This example describes the synthesis of a copolymer that may be used as an intermediate in the synthesis of an emulsifying agent according to an embodiments of the present invention.

### Synthesis of Poly(1,3-propanediolglycerol)

A clean, dry 4-necked round bottom flask equipped with a condenser and overhead stirrer was first charged with 500 g of poly(1,3-propanediol), produced as described in Example 1, and 344 g triglycerol, followed by 2.54 g of 50% sodium hydroxide. The condenser was cooled with tap water at 55-85° F. The reaction was stirred with nitrogen purge for 30 minutes at room temperature before heating the reaction to 360° F. The reaction was held at 360° F. for 3.5 hours and then was cooled to room temperature.

822 grams of a liquid poly(1,3-propanediolglycerol) were collected. The product had the following properties: gel permeation chromatography (GPC) Mp=707, viscosity @25° C.=810 cp, refractive index=1.46998, and hydroxyl value=479 mg KOH/g.

### Example 3

This example describes the synthesis of a fatty acid ester which has a high HLB and which can be used as an E-additive according to an embodiment of the present invention.

### Synthesis of Poly(1,3-propanediolglycerol) mono-distearate

A clean, dry 4-necked round bottom flask equipped with a condenser and overhead stirrer was charged with 171 g of poly(1,3-propanediolglycerol), produced as described in Example 2 above, and 112 g of 70% stearic acid. The condenser was cooled with tap water at 55-85° F. The reaction mixture was heated to 180° F. and stirred until all of the stearic acid was in solution. The reaction mixture was then charged with 1.2 g of 50% sodium hydroxide. The reaction mixture was stirred with nitrogen purge for 30 minutes at 180° F. and the temperature of the reaction mixture was raised to 400° F. The mixture was heated at 400° F. for 2 hours and then cooled. After the reaction mixture was cooled to 180° F., the mixture was neutralized to pH=7 with 2.5 g 70% methane sulfonic acid (MSA). The reaction mixture was filtered through 25 micron filter paper as 218 grams of the resulting solid poly(1,3-propanediolglycerol) mono-distearate product were collected.

The product had the following properties: viscosity @50° C.=69 cp, hydroxyl value=296.5 mg KOH/g, saponification value (SAP)=96.3 mg, HBL=9.5, and Gardner Color—2.5.

### Example 4

This example describes the synthesis of a fatty acid ester which has a low HLB and which can be used as an E-additive according to an embodiment of the present invention.

### Synthesis of Poly(1,3-propanediol) stearate

A clean, dry 4-necked round bottom flask equipped with a condenser and overhead stirrer was charged with 200 g of poly(1,3-propanediol), produced as described in Example 1, and 142 g of 70% stearic acid. The condenser was cooled with tap water at 55-85° F. The reaction mixture was heated to 180° F. and stirred until all of the stearic acid was in solution. The reaction mixture was then charged with 0.88 g of 50% sodium hydroxide and 0.2 g of 70% phosphorous acid. The reactor was stirred with nitrogen purge for 30 minutes at 180° F. and the temperature of the reaction mixture was raised to 400° F. for 6.5 hours and then cooled. After the reaction mixture was cooled to 180° F., the reaction mixture was neutralized to pH=7 with 1 g 85% phosphoric acid. The reaction mixture was filtered through a 25 micron filter and 248 g of a solid poly(1,3-propanediol)stearate were collected. This product had the following properties: viscosity @50° C.=31 cp, hydroxyl value=198.3 mg KOH/g, saponification value (SAP)=91.3 mg KOH/g, HLB=2.3, and Gardner Color—1.0.

### Example 5

This example, like Example 4, describes the synthesis of a fatty acid ester which has a low HLB and which can be used as an E-additive according to an embodiment of the present invention.

### Synthesis of 1,3-propanediol stearate

A clean, dry 4-necked round bottom flask equipped with a condenser and overhead stirrer was charged with 76 g of 1,3-propanediol and 284 g 70% stearic acid. The condenser was cooled with tap water at 55-85° F. The reaction mixture was heated to 180° F. and stirred until all of the stearic acid was in solution. The reaction mixture was then charged with 0.63 g of 50% sodium hydroxide and 0.145 g 70% phosphorous acid. The reaction was stirred with nitrogen purge at 180° F. for 30 minutes and the temperature of the reaction mixture was raised to 360° F. The mixture was heated at 360° F. for 11 hours and then cooled to 180° F., Then the reaction mixture was neutralized to pH=7 with 0.5 g 85% phosphoric acid. The reaction mixture was filtered through a 25 micron filter and 297 g of solid poly(1,3-propanediol)stearate were collected. This product had the following properties: viscosity @70C=10 cp, hydroxyl value=321 mg KOH/g, saponification value (SAP)=176.4 mg, HLB=3.0, and Gardner Color—2.

### Example 6

This example describes the synthesis of a 1,3 -propanediol capped polyglycerol that can be used as an intermediate in the synthesis of an emulsifying agent according to an embodiment of the present invention.

### Synthesis of 1,3-propanediol capped triglycerol

A clean, dry 4-necked round bottom flask equipped with a condenser and overhead stirrer was first charged with 108 g of 1,3-propanediol and 375 g triglycerol, followed by 2.47 g of 50% sodium hydroxide. The condenser was cooled with tap water at 55-85° F. The reaction mixture was stirred with nitrogen purge for 20 minutes at room temperature before heating the mixture to 380° F. The mixture was held at 380° F. for 6.5 hours, and then was cooled to room temperature.

426 g of liquid 1,3-propanediol capped triglycerol were collected. The product had the following properties: viscosity @25° C.=3683 cp, refractive index=1.48382, hydroxyl value=1171 mg KOH/g.

### Example 7

This example describes the synthesis of a fatty acid ester which has a high HLB and which can be used as an E-additive according to an embodiment of the present invention.

### Synthesis of 1,3-propanediol capped triglycerol mono-distearate

A clean, dry 4-necked round bottom flask equipped with a condenser and overhead stirrer was charged with 142 g of 1,3-propanediol capped triglycerol, produced as described in Example 6, and 236 g of 70% stearic acid. The condenser was cooled with tap water at 55-85° F. The reaction mixture was heated to 200° F. and stirred until all of the stearic acid was in solution and was then purged with nitrogen for 20 min. Thereafter, the reaction mixture was charged with 0.7 g of 50% sodium hydroxide and 0.18 g 70% phosphorous acid. The temperature of the reaction mixture was raised to 400° F. and heated for 2.5 hours and then cooled.

320 g of (solid) unneutralized 1,3-propanediol capped mono-distearate were collected. The product had the following properties: hydroxyl value=272 mg KOH/g, saponification value (SAP)=153 mg KOH/g, HLB=9.5, Gardner Color—3.

There follows a description of five examples of personal care formulations which comprise oil-in-water emulsions that contain fatty acid esters that are within the scope of the present invention. Each of the five formulations (A through E) contained the following ingredients (weight %): (1) 81.15% DI water; (2) 0.10% Disodium EDTA; (3) 3.00% glycerol; (4) 0.25% xanthan gum; (5) 1.50% cetearyl alcohol (Lipovol® SC); (6) 8.00% Lipocol® MOS-70(blend of tridecyl stearate, neopentyl glycol discaprylate/dicaprate, tridecyl trimellitate at 45%, 45%, 10% respectively; and (7) 1.00%: Liposerve® PP (phenoxyethanol and parabans). In addition to the above ingredients, each of the five formulations contained also the E-additives as identified below.
(i) Formulation (A): 1.50% of the E-additive of Example 3 above and 3.5% poly(1,3-propanediolglycerol) tristearate having the following characteristics: hydroxyl value 208; saponification value 125; viscosity 50% at 50° C.; HLB (estimated)=7.7; Gardner Color 2. Forumuation (A) had a pH of 6.27 and a viscosity (TF@1.5 rpm at room temperature) of 49370 cp.
(ii) Formulation (B): 1.5% of the E-additive of Example 3 above and 3.5% of poly(1,3-propanediolglycerol) tetrastearate having an SAP of 130; Formulation (B) had a 10 H of 6.2 and a viscosity (TF@1.5 rpm at room temperature) of 8125 cp.
(iii) Formulation (C): 1.5% of the E-additive of Example 3 above and 3.5% of poly(1,3-propanediolglycerol) tribehenate having an SAP of 68; Formulation (e) had a pH of 6.2 and a viscosity (TF@1.5 rpm at room temperature) of 8750 cp.
(iv) Formulation (D): 1.5% of the E-additive of Example 3 above; and 3.5 wt % of poly(1,3-propanediolglycerol) tricocoate having an SAP of 140; Formulation (D) had a pH of 6.1 and a viscosity (TF@1.5 rpm at room temperature) of 2500 c/p.
(v) Formulation (E): 1.5% of the E-additive of Example 3 above and 3.5% of sisterna SP30C sucrose distearate having a melting point of 53-61° C. and an HLB of 6; Formulation(s) had a pH of 6.0 and a viscosity (TF@1.5 rpm at r.t) of 190,200 cp.

There follows a description of four additional examples of personal care formulations (Formulations (F) to (i) which have the same content of ingredients that are present in each of Formulations (A) to (E) above, but which have different E-additives as identified in Table 1 below as (a) to (e).
(a) Poly(1,3-propanediolglycerol) mono-stearate having the following characteristics: SAP=67 and a viscosity @ 50° C. of 50 cp.
(b) Poly(1,3-propanediol) stearate having the following characteristics: SAP=91 and a viscosity @ 50° C. of 31 cp.
(c) Poly(1,2-propanediolglycerol) mono-distearate having the following characteristics: SAP=96 and a viscosity @50° C. of 67 cp.
(d) Poly(1,3-propanediol) stearate having the following characteristics: SAP=102 and a viscosity at 50° C. of 26 cp.
(e) 1,3-(propanediol) stearate having the following characteristics=SAP=176 and a viscosity @ 70° C. of 10 cp.

TABLE 1

| Formulation | E-Additives Wt. % | pH | Viscosity |
|---|---|---|---|
| (F) | 1.5% of (a) and 3.5% of (b) | 5.8 | 36,250 |
| (G) | 1.5% of (c) and 3.5% of (b) | 5.8 | 150,000 |
| (H) | 1.5% of (c) and 3.5% of (d) | 5.95 | 156,200 |
| (I) | 1.5% of (c) and 3.5% of (e) | 4.90 | 201.800 |

The viscosity values are those of homogenized emulsions as evaluated by TF@1.5 rpm at room temperature.

Table 2 below describes an example of a personal care formulation which is in the form of an emulsion and which can be formulated to include a mixture of two E-additives, for example, the fatty acid esters of Examples 3 and 5 above.

TABLE 2

| | Weight | Se- |
|---|---|---|
| Ingredient | Percent | quence |
| Deionized Water | 69.65 | 1 |
| Disodium EDTA (119 - Ruger Chemical Co., Inc.) | 0.10 | 1 |
| Glycerin (119) | 1.00 | 1 |
| Butylene Glycol (119) | 1.00 | 1 |
| Xanthan Gum | 0.25 | 1 |
| Sodium Chloride | 0.75 | 1 |
| Microcrystalline Wax | 1.00 | 2 |
| Cetearyl Alcohol, Lipocol ® SC (1 - Lipo | 1.00 | 2 |
| Chemicals, Inc.) | | |
| Butyrospermum Parkii (Shea Butter), Lipobutter ® | 3.00 | 2 |
| Refined Shea (1) | | |
| Argania Spinosa Kernel Oil, Lipovol ® ARGAN (1) | 2.00 | 2 |
| Glyceryl Stearate and PEG-100 Stearate, | 2.75 | 2 |
| Lipomulse ® 165 (1) | | |
| Caprylic/Capric Triglyceride, Liponate ® GC | 10.00 | 2 |
| Dimethicone | 1.00 | 2 |
| Sorbitan Stearate, Liposorb ® S (1) | 3.50 | 2 |
| Lipo ® Stearic Acid (1) | 3.00 | 3 |
| Preservative | qs | 4 |

To the ingredients identified in Table 2 above, there are added the E-additive of Example 3 above in an amount such that its concentration in the Formulation is about 1.4 wt.%; and the E-additive of Example 5 above is added in an amount such that its concentration in the Formulation is about 3.43 wt. %.
The following procedure can be used to prepare the emulsion which is described in Table 2.
(A) With reference to the ingredients identified in Table 2 by “Sequence 1”, they are added to a main vessel and mixed with a medium speed propeller and heated to 75° C. to provide a mixture.
(B) The ingredients identified in Table 2 by “Sequence 2” and the E-additives are added to a side vessel and mixed as they are heated.
(C) The resulting mixture of “Sequence 2” is added to the mixture of “Sequence 1” when its temperature reaches 75° C. The composition of the combined mixtures is stirred with medium mixing as cooling is begun.
(D) When the temperature of the composition reaches 60° C., the stearic acid of “Sequence 3” is added with continued mixing.
(E) When the temperature of the resulting composition is 45° C., the preservative of “Sequence 4” is added with continued mixing. When the involved temperature reaches 32° C., preparation of the emulsion can be considered complete.

Formulation J below is an example of an all natural body lotion that feels soft and elegant and leaves the skin smooth and moisturized.

TABLE 3
Formulation J

| | Weight | Se- |
|---|---|---|
| Ingredient | Percent | quence |
| Deionized Water | 82.90 | 1 |
| Disodium EDTA (119 - Ruger Chemical Co., Inc.) | 0.10 | 1 |
| Xanthan Gum (119) | 0.25 | 2 |
| Glycerin 99.7% (119) | 2.50 | 2 |
| Propanediol Stearate, Lipomulse ® ECO L (1 - Lipo | 2.45 | 3 |
| Chemicals, Inc.) | | |
| Poly (Glycerin/Propandiol) Stearate, Lipomulse ® | 1.05 | 3 |
| ECO H (1) | | |
| Crambe Abyssinica Seed Oil, Lipovol ® Crambe (1) | 2.75 | 3 |
| Vitis Vinifera (Grape) Seed Oil, Lipovol ® G (1) | 4.25 | 3 |
| Simmondsia Chinensis (Jojoba) Butter, | 2.00 | 3 |
| Iso Jojoba ® 35 (144 - Lipo Chemicals - Desert | | |
| Whale Jojoba) | | |
| Cetearyl Alcohol, Lipocol SC (1) | 1.75 | 3 |
| Preservative | qs | 4 |

The following procedure can be used to prepare the emulsion which is described in Table 3.

(A) In a main vessel, the ingredients of “Sequence 1” are stirred with a medium speed propeller to form a mixture.

(B) Slowly add premixed ingredients of “Sequence 2” to the mixture of (A) while mixing and heating to a temperature of 80° C. and maintain the combined mixtures at 80° C. for 30 minutes.

(C) Start heating the ingredients of “Sequence 3” while mixing and heating the mixture to a temperature of 80° C. and add it to the combined mixtures of (A) and (B) in the main vessel while mixing.

(D) After 5-10 minutes, homogenize the composition in the main vessel at 4000 rpm for 3-5 minutes at a temperature of 70 to 80° C.

(E) Thereafter switch to propeller mixing and start to slowly cool the homogenized composition.

(F) When the temperature approaches 45° C., add to the homogenized composition the preservative of Sequence 4 with, continued mixing.

(G) When temperature of the homogenized composition reaches 35° C., the preparation of the homogenized emulsion can be considered complete.

Formulation K below is an example of a soft all natural cream that conditions and leaves the skin moisturized and vibrant.

TABLE 4
Formulation K

| | Weight | Se- |
|---|---|---|
| Ingredient | Percent | quence |
| Deionized Water | 72.05 | 1 |
| Disodium EDTA (119 - Ruger Chemical Co., Inc.) | 0.10 | 1 |
| Xantham Gum (119) | 0.25 | 2 |
| Glycerin 99.7% (119) | 2.00 | 2 |
| Polypropanediol Stearate, Lipomulse ® ECO L2 (1- | 3.50 | 3 |
| Lipo Chemicals, Inc.) | | |
| Poly(Glycerin/Propandiol) Stearates, Lipomulse ® | 1.50 | 3 |
| ECO H (1) | | |
| Cetyl Esters, Liponate ® SPS-VEG (1) | 2.50 | 3 |
| Cocos Nucifera (Coconut) Oil, Lipovol ® C76 (1) | 5.00 | 3 |
| Olea Europea (Olive) Fruit Oil, Lipovol ® O (1) | 4.00 | 3 |
| Simmondsia Chinensis (Jojoba) Seed Oil, DW | 6.00 | 3 |
| Jojoba Colorless (144 Lipo Chemicals - Desert | | |
| Whale Jojoba | | |
| Cetearyl Alcohol, Lipocol ® SC (1) | 3.0 | 3 |
| Tocopherol, Vitamin E | 0.10 | 3 |
| Preservative | qs | 4 |

The following procedure can be used to prepare the emulsion which is described in Table 4.

(A) In a main vessel, the ingredients of “Sequence 1” are stirred with a medium speed propeller to form a mixture.

(B) Slowly add premixed ingredients of “Sequence 2” to the mixture of (A) while mixing and heating to a temperature of 80° C. and maintain the mixture at 80° C. for 30 minutes.

(C) Start heating the ingredients of “Sequence 3” while mixing and heating to a temperature of 80° C. and add the heated mixture to the combined mixtures of (A) and

(B) above in the main vessel while mixing.

(D) After 5-10 minutes, homogenize the composition in the main vessel at 4000 rpm for 3-5 minutes at temperature of 70 to 80° C.

(E) Thereafter, switch to propeller mixing and start to slowly cool the homogenized composition.

(F) When its temperature approaches 45° C., add the preservative of “Sequence 4” with continued mixing to the homogenized composition.

(G) When the temperature of the homogenized composition reaches 35° C., preparation of the homogenized emulsion can be considered complete.

Formulation L below is an example of a rich body butter that pampers the skin leaving it smooth and moisturized.

TABLE 5
Formulation L

| | Weight | Se- |
|---|---|---|
| Ingredient | Percent | quence |
| Deionized Water | 70.05 | 1 |
| Disodium EDTA (119- Ruger Chemical Co. Inc) | 0.10 | 1 |
| Xantham Gum (119) | 0.25 | 2 |
| Glycerin 99.7% (119) | 3.50 | 2 |
| Polypropanediol Stearate, Lipomulse ® ECO L2 (1- | 3.50 | 3 |
| Lipo Chemicals, Inc.) | | |
| Poly(Glycerin/Propandiol) Stearates, Lipomulse ® | 1.50 | 3 |
| ECO H (1) | | |
| Cetyl Esters, Liponate ® SPS-VEG (1) | 3.00 | 3 |
| Persea Gratissima (Avocado) Oil, Lipovol ® A (1) | 5.00 | 3 |
| Vitis Vinifera (Grape) Seed Oil, Lipovol ® G (1) | 4.00 | 3 |
| Orbignya Oleifera Seed Oil, Lipobutter ® Refined | 6.00 | 3 |
| Mango (1) | | |
| Cetearyl Alcohol, Lipocol ® SC (1) | 3.00 | 3 |
| Tocophearol, Vitamin E | 0.10 | 3 |
| Preservative | qs | 4 |

The following procedure can be used to prepare the emulsion which is described in Table 5.

(A) In a main vessel, the ingredients of “Sequence 1” are stirred with a medium speed propeller to form a mixture.

(B) Slowly add premixed ingredients of “Sequence 2” while mixing and heating to a temperature of 80° C. and maintain the mixture at 80° C. for 30 minutes.

(C) Start heating the ingredients of “Sequence 3” while mixing and heating to a temperature of 80° C. and add the heated mixture to the combined mixtures of (A) and (B) above in the main vessel while mixing.

(D) After 5-10 minutes, homogenize the composition in the main vessel at 4000rpm for 3-5 minutes at a temperature of 70° C. to 80° C.

(E) Thereafter, switch to propeller mixing and start to slowly cool the homogenized composition.

(F) When its temperature approaches 45° C., add the preservative of “Sequence 4” with continued mixing to the homogenized composition.

(G) When temperature of the homogenized composition reaches 35° C., preparation of the homogenized emulsion can be considered complete.

Formulation M below is an example of a natural conditioner that leaves hair soft, smooth and manageable.

TABLE 5
Formulation M

| | Weight | Se- |
|---|---|---|
| Ingredient | Percent | quence |
| Deionized Water | 82.90 | 1 |
| Tetrasodium EDTA (119 - Ruger Chemical Co., | 0.10 | 1 |
| Inc.) | | |
| Hydroxyethylcellulose, Hatrasol 250HHRCS (119) | 0.25 | 2 |
| Glycerin 99.7% (119) | 2.50 | 3 |
| Cetrimonium Chloride | 3.0 | 3 |
| Propanediol Stearate, Lipomulse ® ECO L (1 - Lipo | 3.5 | 4 |
| Chemicals Inc.) | | |
| Poly(Glycerin/Propanediol) Stearates, Lipomulse ® | 1.50 | 4 |
| ECO H (1) | | |
| Cetyl Esters, Liponate ® SPS Veg (1) | 1.00 | 4 |
| Argania Spinoso Kernel Oil, Lipovol ® Argan (1) | 1.50% | 4 |
| Persea Gratissma Avacado Oil, Lipovol ® AL) | 5.00 | 3 |
| Orbignya Oleifera Seed Oil, Lopibutter ® Refined | 1% | 4 |
| Mango (1) | | |
| Cetearyl Alcohol, Lipovol ® SC (1) | 3.00 | 4 |
| Preservative | qs | 4 |

The following procedure can be used to prepare the emulsion which is described in Table 5.

(A) In a main vessel, the ingredients of “Sequence 1” are stirred with a medium speed propeller to form a mixture.

(B) Slowly add premixed ingredients of “Sequence 2” to the mixture of (A) while mixing and heating to a temperature of 80° C. and maintain the mixture at 80° C. for 30 minutes.

(C) Start heating the ingredients of “Sequence 3” while mixing and heating to a temperature of 80° C. and add the heated mixture to the combined mixtures of (A) and (B) in the main vessel while mixing.

(D) After 5-10 minutes, homogenize the composition in the main vessel at 4000 rpm for 3-5 minutes at a temperature of 70 to 80° C.

(E) Thereafter, switch to propeller mixing and start to slowly cool the homogenized composition.

(F) When the temperature approaches 45° C., add the preservative of “Sequence 4” to the homogenized composition with continued mixing.

(G) When temperature reaches 35° C., the preparation of the homogenized emulsion can be considered complete.

In summary, the present invention provides an emulsifying agent that can be used to environmental advantage in numerous and diversified products.

We claim:

1. A fatty acid ester in which the source of the acid residue of the ester is a fatty acid having a chain length of about 2 to about 40 carbon atoms (hereafter “a fatty acid”) and the source of the alcohol residue of the ester is one or more of:
(A) 1,3-propanediol;
(B) poly(1,3-propanediol) polymer;
(C) 1,3-propanediol glycerol;
(D) poly(1,3-propanediol glycerol) copolymer; or
(E) 1,3-propanediol capped glycerol.

2. A fatty acid ester according to claim 1 including a fatty acid selected from the group consisting of stearic acid, coco fatty acid, oleic acid, behenic acid.

3-17. (canceled)
